(19) United States
(12) Patent Application Publication
(10) Pub. No.: US 2008/0068182 A1
(43) Pub. Date: Mar. 20, 2008

(54) SENSOR FOR MEASURING RELATIVE CONDUCTIVITY CHANGES IN BIOLOGICAL TISSUE

(76) Inventors: Brian Watson, Gainesville, FL (US); Jeffrey Ridgway, San Diego, CA (US); Patrick Castelaz, Yorba Linda, CA (US)

Correspondence Address: NYDEGGER & ASSOCIATES, 348 OLIVE STREET, SAN DIEGO, CA, 92103, US

(21) Appl. No.: 11/531,629

(22) Filed: Sep. 13, 2006

(51) Int. Cl. G08B 23/00; A61B 5/00; G08B 1/08

(52) U.S. Cl. 340/573.1; 340/539.12; 600/300

(57) ABSTRACT

A sensor for detecting changes in electrical conductivity in a patient is disclosed. The sensor includes a transmit antenna that has an interior area bounded by a periphery. Importantly, the periphery includes an outer ring portion and an inner ring portion therein that partially surrounds an open area. Further, the sensor includes a receive antenna that has a substantially circular circumference that bounds an interior area. In order to balance the antennas, approximately half of the interior area of the receive antenna is superposed on the interior area of the second antenna. In operation, the antennas are positioned at a selected distance from one another near a patient. Then a signal is sent from the transmit antenna to the receive antenna. The signal is affected by the conductivity of the patient. As a result, the received signal may be monitored to detect electrical conductivity changes in the patient.

### FIELD OF THE INVENTION

The present invention pertains generally to an electromagnetic impedance sensor. More particularly, the present invention pertains to a sensor that monitors a patient's respiratory and heart rate by detecting changes in electrical conductivity. The present invention is particularly, but not exclusively, useful as a sensor for detecting an onset of respiratory distress in a patient.

### BACKGROUND OF THE INVENTION

Mammals' bodies are comprised mainly of water with an average conductivity and relative dielectric constant that is approximately that of seawater. Further, these parameters vary significantly for each of the body's organs. As a result of this variance, movement or deformation of the shape of a body results in changes in the electrical conductivity of the body. For instance, the change in shape of a body during respiration causes a measurable change in electrical conductivity. Further, during the cardiac cycle, the volume of blood in each chamber of the heart varies. Accordingly, a periodic change in the electrical conductivity of the heart is produced.

It is known that changes in electrical conductivity can be electronically monitored by observing changes in electromagnetic impedance. For this purpose, it is known that a sensor having a transmitting coil and a receiving coil can measure changes in electrical impedance. To do this, a signal is sent along the transmitter coil at a selected frequency to create an electromagnetic field. The electromagnetic field then interacts with any conductive materials in the environment, e.g., blood. In turn, this causes the conductive materials to generate currents with weak magnetic fields of their own. In response to these weak magnetic fields, currents are created in the receiving coil that form a received signal which can then be analyzed.

Changes in the respiration and heart rate of patients, including infants, may signal the onset of respiratory distress. For example, in the case of infants, it is known that respiratory distress is an early symptom of Sudden Infant Death Syndrome (SIDS). Therefore, the identification of respiratory distress in an infant can lead to a timely medical intervention that may prevent a SIDS death. Also, the identification of respiratory distress in adult patients may be helpful in diagnosing various afflictions. In any case, the long-term chronic use of current electromagnetic impedance systems, in which electrodes are placed on the patient's chest, may be impractical. This is particularly so for infants. In addition, currently used systems typically measure impedance in the entire thorax rather than locally (i.e. lungs, heart). Thus, they inherently lack a level of precision that may be useful.

In light of the above, it is an object of the present invention to provide an electromagnetic impedance sensor, and a method, that can be used to periodically measure the heart and respiration rate of an infant or other patient, without direct contact with the patient. It is another object of the present invention to provide a precise sensor and method that measures local electromagnetic impedance near the heart instead of in the entire thorax. It is another object of the present invention to provide a precise sensor and method that emits electromagnetic radiation well below IEEE limits for exposure. Yet another object of the present invention is to provide a sensor and a method for detecting changes in electrical conductivity in a patient which avoids false alarms. Still another object of the present invention is to provide sensors and methods for their manufacture which are easy to use, relatively simple to implement, and comparatively cost effective.

### SUMMARY OF THE INVENTION

The present invention is directed to a sensor for detecting changes in overall electrical conductivity in an environment. More specifically, the sensor of the present invention detects changes in electrical conductivity of a mammal's body. For instance, the sensor may monitor a patient (e.g. an infant) to detect electrical conductivity changes that are indicative of an onset of respiratory distress. The sensor of the present invention includes a transmit antenna having a substantially crescent-shaped periphery for transmitting a signal. Specifically, the transmit antenna includes a substantially circular outer ring portion, with a cutout portion consisting of a substantially circular inner ring portion. The periphery of the transmit antenna bounds an interior area and substantially surrounds an open area that is partially bordered by the inner ring portion.

Further, the sensor comprises an antenna for receiving the transmitted signal. This receive antenna has a substantially circular circumference that bounds an interior area. For purposes of the present invention, the receive antenna is positioned relative to the transmit antenna so that approximately half of the receive antenna's interior area is superposed on the transmit antenna's interior area. Consequently, the remaining half of the receive antenna's interior area is outside the transmit antenna's interior area. Stated differently, approximately half of the receive antenna's interior area is superposed on the open area that is partially bordered by the inner ring portion of the transmit antenna. As a result of this arrangement, the total magnetic flux through the receive antenna is zero.

Structurally, both antennas consist of flat wire and are preferably printed circuit board antennas. Further, the flat wire of the transmit antenna is formed with three turns while the flat wire of the receive antenna is formed with six turns. For each antenna, the flat wire has the same constant width. Also, in both antennas each turn in the flat wires is distanced from an adjacent turn by a distance equal to half of the wire width to reduce the parasitic capacitance between turns.

For the present invention, the sensor further includes electronic components for supplying an excitation signal to the transmit antenna. Further, the sensor includes electronic components for supplying an adjusted signal to the receive antenna to dynamically balance the sensor to compensate for changes in the environment. Also, the sensor includes electronic components for recording and analyzing the adjusted and received signals to detect changes in the electrical conductivity around the antennas.

During preparation of the sensor, an excitation signal is supplied to and emitted by the transmit antenna. Preferably, this signal is a continuous wave sinusoidal signal with a frequency of approximately 5 MHz. In response, a signal is created in the receive antenna. During balancing, the position between the antennas is adjusted until the constant portion of the received signal is minimized. Thereafter, the sensor is ready for use.

During use, the antennas are positioned near the patient and the excitation signal is emitted by the transmit antenna. Again, a signal is created in the receive antenna. Analysis of the received signal results in the detection of changes in the electrical impedance and conductivity of the patient. Further, the electrical impedance and conductivity are used to identify the respiratory and heart rate of the patient, as well as any onset of respiratory distress.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a perspective view of a sensor for detecting the onset of respiratory distress in an infant in accordance with an embodiment of the present invention;

FIG. 2 is an overhead view of a transmit antenna of the sensor shown in FIG. 1 in accordance with the present invention;

FIG. 3 is an overhead view of a receive antenna of the sensor shown in FIG. 1 in accordance with the present invention;

FIG. 4 is a schematic view of the receive antenna of FIG. 3 partially superposed on the transmit antenna (shown in phantom) of FIG. 2 in accordance with the present invention; and

FIG. 5 is a block diagram of a system for detecting changes in electrical conductivity.

### DESCRIPTION OF THE PREFERRED EMBODIMENTS

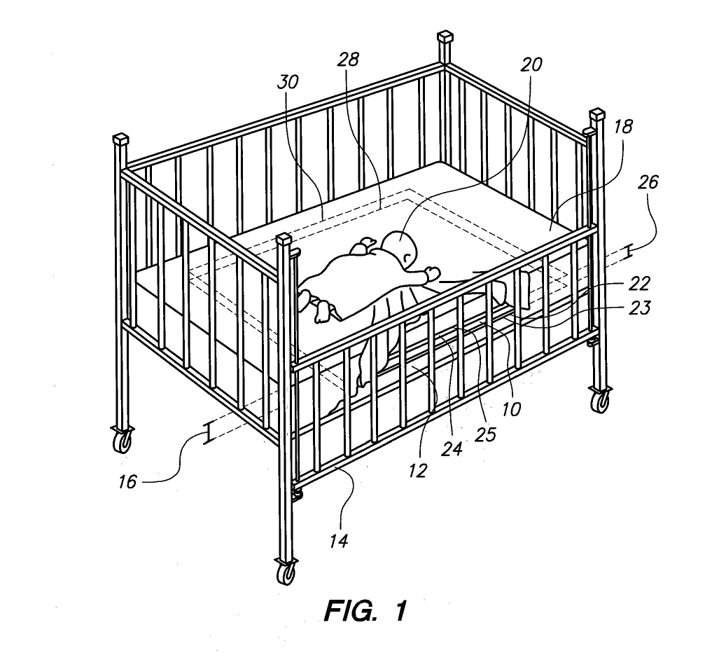

Referring initially to FIG. 1, a sensor for detecting changes in electrical conductivity in an environment is shown and designated 10. For exemplary purposes, the sensor 10 is mounted within a mattress 12 in a crib 14 at a predetermined distance 16 from the surface 18 of the mattress 12. Further, it can be seen that an infant 20 is sleeping on the surface 18 of the mattress 12. As shown, the sensor 10 includes a transmit antenna 22 mounted on a board 23 and a receive antenna 24 mounted on a board 25. The antennas 22, 24 are separated by a selected distance 26. Specifically, the antenna 22 defines a plane 28, and the antenna 24 defines a substantially parallel plane 30 at the selected distance 26 from the plane 28. For purposes of the present invention, the antenna 22 transmits a signal that is received by the antenna 24. Thereafter, the received signal is monitored and electromagnetic impendence detection is used to detect changes in the electrical conductivity of the environment, e.g., the infant's body 20 in the illustrated example. Further, the sensor 10 monitors the electrical conductivity changes to identify the onset of respiratory distress in the infant 20.

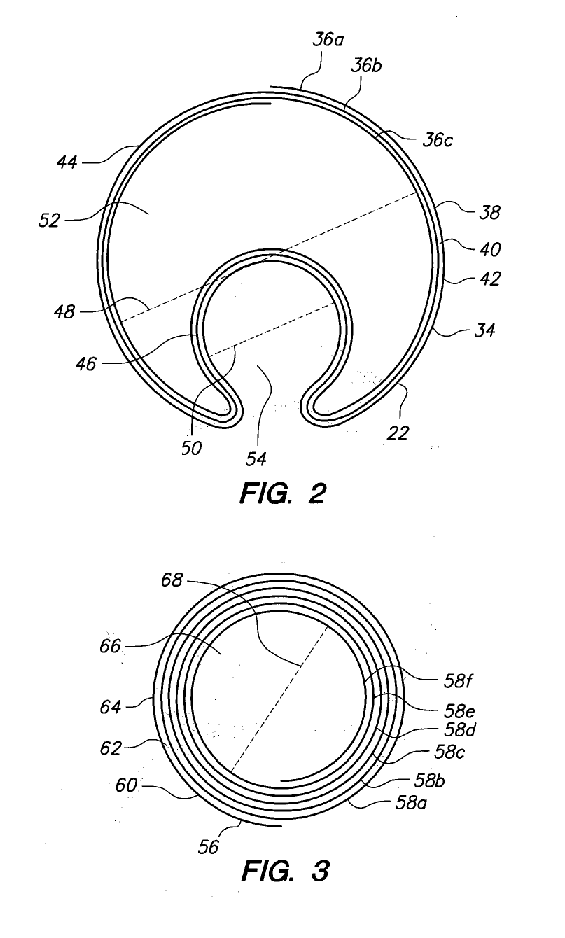

Referring now to FIG. 2, the structure of the transmit antenna 22 is illustrated. As shown, the transmit antenna 22 comprises a substantially flat wire 34 that forms three spiral turns 36a, 36b, 36c. To allow for precise control of the geometry of the antenna 22, the wire 34 is preferably a printed circuit board. Also, the transmit antenna 22 is preferably a broadband series resonant antenna with self-resonance of approximately 14 MHz. Structurally, the wire 34 has a constant width 38 of about 200 mils and a height or depth of about 2.6 mils. Further, each turn 36a-c of the wire 34 is separated from the adjacent turn 36a-c by a distance 40 that is equal to one half the width 38 of the wire 34. (FIG. 2 is not drawn to scale.) These geometric dimensions have been determined to reduce the parasitic capacitance between turns 36a-c.

As shown in FIG. 2, the antenna 22 has a substantially crescent-shaped periphery 42, i.e., the periphery 42 has the shape of a large circle with a portion of a smaller circle removed from its edge. Specifically, the antenna 22 is formed with an outer ring portion 44 and an inner ring portion 46 that are both substantially circular. As shown, the outer ring portion 44 defines an outer diameter 48 of about twelve inches and the inner ring portion 46 defines an inner diameter 50 of about six inches. Further, the periphery 42 bounds an interior area 52. As shown, the inner ring portion 46 of the periphery 42 borders an open area 54 that is partially surrounded by the antenna 22.

Referring now to FIG. 3, the structure of the receive antenna 24 is illustrated. As shown, the receive antenna 24 comprises a substantially flat wire 56 that forms six spiral turns 58a-f. As with the transmit antenna 22, the wire 56 of the receive antenna 24 preferably is a printed circuit board. Further, the wire 56 has a constant width 60 that is equal to the width 38. Also, each turn 58a-f of the wire 56 is separated from the adjacent turn 58a-f by a distance 62 that is equal to one half the width 60 of the wire 56. (FIG. 3 is not drawn to scale.) As with the transmit antenna 22, these geometric dimensions have been determined to reduce the parasitic capacitance between turns 58.

As shown in FIG. 3, the antenna 24 has a substantially circular circumference 64 which bounds an interior area 66 of the antenna 24. For the present invention, the circumference 64 defines an average antenna diameter 68 of about six inches. Preferably, the receive antenna 24 is a parallel resonant antenna with self-resonance frequency of approximately 12 MHz and a quality factor of approximately 100.

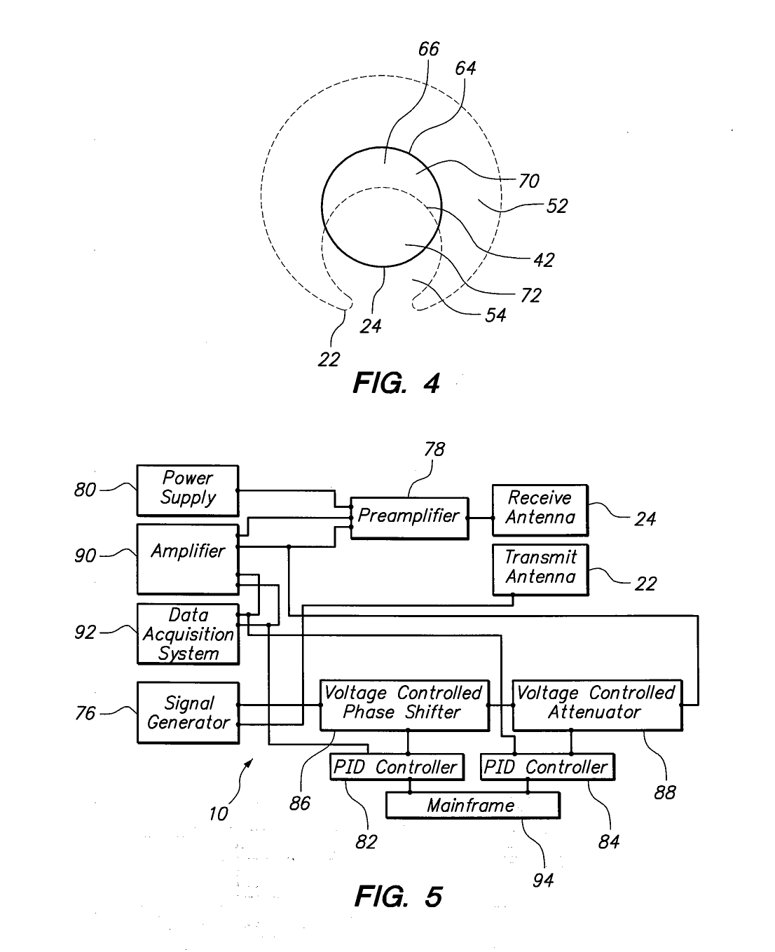

Referring now to FIG. 4, the relationship between the transmit antenna 22 (shown in phantom) and the receive antenna 24 is schematically illustrated. For purposes of the present invention, the receive antenna 24 is positioned substantially parallel to, and at a selected distance 26 (shown in FIG. 1) from, the transmit antenna 22. As shown in FIG. 4, a portion 70 of the interior area 66 of the receive antenna 24 is superposed on the interior area 52 of the transmit antenna 22 while a portion 72 of the interior area 66 is superposed on the open area 54. For purposes of the present invention, each portion 70 and 72 is equal to approximately half of the interior area 66. In other words, when the periphery 42 of the transmit antenna 22 is extended perpendicular from the plane 28 (shown in FIG. 1) through the receive antenna 24, the periphery 42 divides the interior area 66 of the receive antenna 24 into substantially equal portions 70 and 72. This arrangement results in zero total magnetic flux through the receive antenna 24.

Turning now to FIG. 5, the transmit antenna 22 and the receive antenna 24 of the sensor 10 are shown in connection with other electronic components for detecting changes in electrical conductivity. Referring first to the transmit side of the system, the transmit antenna 22 is shown connected to a signal generator 76. Preferably, the generator 76 has a superior spurious free dynamic range above 90 dB. For purposes of the present invention, the generator 76 supplies the transmit antenna 22 with an excitation signal that is preferably a continuous wave sinusoidal signal in the low MHz range.

In FIG. 5, the receive antenna 24 is shown connected to a preamplifier 78. Further, the preamplifier 78 is connected to receive power from a power supply 80 such as a battery (not shown). Also, the preamplifier 78 is connected to the generator 76 through two proportional integral derivative (PID) controllers 82, 84. For the present invention, the PID controllers 82, 84 are shown in connection with a voltage controlled phase shifter 86 and a voltage controlled attenuator 88. This arrangement allows the PID controllers 82, 84 to continuously adjust the amplitude and phase of the signal sent to the preamplifier 78 from the generator 76. As shown, the system includes an amplifier 90 that receives the adjusted signal from the PID controllers 82, 84 for reference purposes.

Still referring to FIG. 5, the amplifier 90 is also connected to the preamplifier 78 to receive the preamplifier output signal. Taken in consideration with its connection to the PID controllers 82, 84, it can be seen that the amplifier 90 receives both the output signal (from the preamplifier 78) and the adjusted signal (from the PID controllers 82, 84). Further, the amplifier 90 is connected to a data acquisition system 92 and to a mainframe 94 through the PID controllers 82, 84.

With this understanding of the components of the sensor 10, the operation of the sensor may be understood. Initially, the electronic components are connected as shown in FIG. 5. Thereafter, the transmit antenna 22 and the receive antenna 24 are placed at a desired position relative to one another. Specifically, the receive antenna 24 is positioned so that approximately half of its interior area 66 is superposed on the interior area 52 of the transmit antenna 22. Accordingly, half of the receive antenna's interior area 66 is superposed on the open area 54 in the transmit antenna 22. Further, the receive antenna 24 is positioned at a selected distance 26 from the transmit antenna 22. Specifically, the distance 26 is selected to balance the sensor 10, i.e., the position of the receive antenna 24 relative to the transmit antenna 22 is adjusted to minimize the output of the receive antenna 24. By minimizing the constant portion of the receive antenna output, the sensitivity of the sensor 10 is increased. Specifically, the necessary dynamic range of the data acquisition system 92 is decreased. In certain embodiments, the board 23 and board 25 may be threadedly engaged to allow movement therebetween. For such embodiments, a thin Mylar sheet may be positioned between the boards 23, 25 to facilitate relative movement therebetween. In practice, the receive antenna 24 is typically positioned directly on top of the transmit antenna 22, such that the selected distance 26 is extremely small. Further, in certain embodiments, the transmit antenna 22 and receive antenna 24 may be printed on a single circuit board at the selected distance 26 so that further mechanical balancing is unnecessary.

After balancing the sensor 10, an adjusted signal is added to the preamplifier 78 from the PID controllers 82, 84. Specifically, the amplitude and phase of this adjusted signal are controlled to minimize the preamplifier output signal and to eliminate drift. The time constant of this adjustment is typically several seconds. When the sensor 10 is operated, the data acquisition system 92 uses the amplitude and the phase of the preamplifier output signal and the adjusted signal in signal processing algorithms to detect changes in the electrical conductivity of the environment around the antennas 22, 24. Therefore, when a body is in the environment, a change in the distribution of any conductive fluid (e.g. blood) in the body produces a change in the antenna impedance and a resultant change in the output signal. In practice, changes in the output signal have been identified as corresponding to respiration as well as movement of blood into different chambers of the heart. Signal processing algorithms can isolate the portion of the signal due to the cardiac cycle and/or due to respiration. In this manner, the sensor 10 may identify if a patient in the environment around the sensor 10 is in respiratory distress. Further, because the impedance of the sensor 10 changes in specific ways when a body is placed in the sensor's environment, the sensor 10 can identify whether the body has been removed from the environment and, therefore, will not cause a false alarm of respiratory distress.

In certain embodiments of the present invention, the sensor 10 may be built into a mattress 12 as shown in FIG. 1. In other embodiments, the sensor 10 may be used in conjunction with a separate platform and placed at a desired position relative to the patient. In still other embodiments, the antennas 22, 24 of the sensor 10 may be printed on flexible boards and placed in a blanket for use directly on the patient. Regardless of the specific construction, it is preferred that the antennas 22, 24 be positioned within several centimeters (less than 12 cm) of the patient during monitoring.

While the particular Planar Sensor for Measuring Relative Conductivity Changes in Biological Tissue as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A sensor for detecting an onset of respiratory distress in a subject which comprises:
a first antenna for transmitting a signal, said first antenna having a periphery that bounds a first interior area, with said periphery including an outer ring portion and an inner ring portion with the inner ring portion substantially surrounding an open area, with said first antenna being positioned at a selected position relative to the subject;
a second antenna for receiving the transmitted signal, said second antenna having a substantially circular circumference bounding a second interior area, wherein approximately half of the second interior area is superposed on the first interior area and approximately half of the second interior area is superposed on the open area; and
a means for monitoring the received signal to detect the onset of respiratory distress in the subject.

2. A sensor as recited in claim 1 wherein the onset of respiratory distress in the subject is detected by monitoring changes in electrical conductivity in the subject.

3. A sensor as recited in claim 1 wherein said first antenna consists of a flat wire forming three turns and wherein said second antenna consists of a flat wire forming six turns.

4. A sensor as recited in claim 3 wherein the first antenna and the second antenna are printed circuit board antennas.

5. A sensor as recited in claim 3 wherein each wire has a width, and wherein each turn is distanced from an adjacent turn by a distance equal to half of the width.

6. A sensor as recited in claim 1 further comprising a means for dynamically balancing the sensor to compensate for changes in the environment.

7. A sensor as recited in claim 1 wherein the signal is a continuous wave sinusoidal signal with a frequency of approximately 5 MHz.

8. A sensor as recited in claim 1 wherein a secondary signal is added to the received signal and wherein the sensor further comprises a proportional integral derivative controller to continuously adjust the secondary signal to eliminate drift in the sensor.

9. A sensor for detecting changes in overall electrical conductivity in an environment which comprises:
a first antenna for transmitting a signal, said first antenna having a substantially crescent shaped periphery, with said first antenna defining a first plane and being selectively positioned relative to the environment;
a second antenna for receiving the transmitted signal, said second antenna having a substantially circular circumference bounding an interior area, with said second antenna being substantially parallel to said first plane, and wherein the periphery of the first antenna divides the interior area of the second antenna into substantially equal portions when the periphery of the first antenna is extended perpendicular from the first plane through the circumference of the second antenna; and
a means for monitoring the received signal to detect changes in overall electrical conductivity in the environment.

10. A sensor as recited in claim 9 wherein the periphery includes an outer ring portion and an inner ring portion, and wherein the inner ring portion of the periphery of the first antenna divides the interior area of the second antenna into substantially equal portions when the periphery of the first antenna is extended perpendicular from the plane through the circumference of the second antenna.

11. A sensor as recited in claim 9 wherein said first antenna consists of a flat wire forming three turns and wherein said second antenna consists of a flat wire forming six turns.

12. A sensor as recited in claim 11 wherein each wire has a width, and wherein each turn is distanced from an adjacent turn by a distance equal to half of the width.

13. A sensor as recited in claim 9 wherein the environment includes a subject and wherein the changes in overall electrical conductivity in the environment are caused by the onset of respiratory distress in the subject.

14. A sensor as recited in claim 9 further comprising a means for dynamically balancing the sensor to compensate for changes in the environment.

15. A sensor as recited in claim 9 wherein the signal is a continuous wave sinusoidal signal with a frequency of approximately 5 MHz.

16. A sensor as recited in claim 9 wherein a secondary signal is added to the received signal and wherein the sensor further comprises a proportional integral derivative controller to continuously adjust the secondary signal to eliminate drift in the sensor.

17. A method for detecting changes in overall electrical conductivity in an environment which comprises:
providing a sensor including a first antenna and a second antenna, with said first antenna having a substantially crescent shaped periphery that bounds a first interior area, and with said second antenna having a substantially circular circumference bounding a second interior area;
placing the first antenna at a selected position relative to the environment;
positioning the second antenna parallel to the first antenna with approximately half of the second interior area being superposed on the first interior area;
transmitting a signal from the first antenna;
receiving the signal with the second antenna; and
monitoring the received signal to detect the changes in overall electrical conductivity in the environment.

18. A method as recited in claim 17 further comprising the step of dynamically balancing the sensor to compensate for changes in the environment.

19. A method as recited in claim 17 further comprising the steps of:
adding a secondary signal to the received signal; and
continuously adjusting the secondary signal to eliminate drift in the sensor.

20. A method as recited in claim 17 wherein the environment includes a subject and the changes in overall electrical conductivity in the environment are caused by changes in electrical conductivity in the subject.
